(19) United States
(12) Patent Application Publication
(10) Pub. No.: US 2025/0058120 A1
(43) Pub. Date: Feb. 20, 2025

(54) INTEGRATION OF FIBER TRACTS INTO DEEP BRAIN STIMULATION TARGETING

(71) Applicant: Boston Scientific Neuromodulation Corporation, Valencia, CA (US)

(72) Inventors: Richard Mustakos, Simi Valley, CA (US); Mahsa Malekmohammadi, Sherman Oaks, CA (US); Stephen Carcieri, Los Angeles, CA (US)

(73) Assignee: Boston Scientific Neuromodulation Corporation, Valencia, CA (US)

(21) Appl. No.: 18/804,599

(22) Filed: Aug. 14, 2024

Related U.S. Application Data
(60) Provisional application No. 63/533,041, filed on Aug. 16, 2023.

(51) Int. Cl. A61N 1/36; A61N 1/05; A61N 1/372; G16H 20/40

(52) U.S. Cl. CPC A61N 1/36139; A61N 1/0534; A61N 1/37247; G16H 20/40

(57) ABSTRACT

Systems and methods for configuring neurostimulation systems to identify optimal therapies for use in a patient. Positional data for a lead implanted in the patient and anatomical data are obtained. Non-linear response structures are identified in the patient, and therapy metrics are generated using non-linear functions of the quantity of volume segments in the non-linear response structures that would be activated by a given therapy configuration including a combination of current fractionalization and current amplitude.

### CROSS REFERENCE TO RELATED APPLICATIONS

The present application claims the benefit of U.S. Provisional Patent Application No. 63/533,041, filed Aug. 16, 2023, titled INTEGRATION OF FIBER TRACTS INTO DEEP BRAIN STIMULATION TARGETING, the disclosure of which is incorporated herein by reference.

### BACKGROUND

Deep brain stimulation (DBS) is a form of neuromodulation in which electrodes are implanted into the brain of a patient and used to deliver stimuli. Therapies directed to a variety of ailments, including Alzheimer's Disease, Parkinson's Disease, cognitive and/or memory decline, depression and other disorders have been proposed and/or implemented. Each patient has unique anatomy, and each treated ailment may call for different portions of the brain to receive therapy. As a result, accurate and precise targeting of therapy is desired.

A white matter nerve tract is a bundle of nerve fibers (axons) connecting nuclei or other elements of the central nervous system. The main nerve tracts in the brain include association fibers, commissural fibers, and projection fibers. Association tracts connect cortical areas within the same cerebral hemisphere. Association tracts may, for example, link perceptual and memory centers of the brain. Commissural tracts connect corresponding cortical areas in the two hemispheres by crossing from one cerebral hemisphere to the other through bridges called commissures, and allow the left and right sides of the cerebrum to communicate with each other. The largest commissure is the corpus callosum, while other, smaller commissural tracts pass through the anterior and posterior commissures. Other commissures are the hippocampal commissure, and the habenular commissure. Projection tracts connect the cerebral cortex with the corpus striatum, diencephalon, brainstem and the spinal cord, and can carry sensory or motor signals. Of particular note for some types of DBS targeting the thalamus and basal forebrain is the internal capsule, located between the thalamus and basal nuclei, carrying fibers which then spread out to connect to specific areas of the cortex.

Some brain structures, such as gray matter structures (mostly comprising neural cell bodies, axon terminals, and dendrites, as well as nerve synapses) respond to neural stimulation in proportion to the quantity or volume of tissue that is activated by a stimulus in what can be characterized as a linear response. However, other structures, such as white matter fiber tracts, respond in a non-linear manner to electric field-induced activation. Moreover, when a fiber tract is activated in whole or in part, this can affect distant neural structures from the location of the stimulus, including the grey matter structures at either end of the activated fiber tract. New and alternative methods and systems for targeting DBS therapy that can account for fiber tracts are desired.

### Overview

The present inventors have recognized, among other things, that a problem to be solved is the need for new and/or alternative methods and systems for targeting DBS therapy that can account for fiber tracts.

A first illustrative and non-limiting example takes the form of a system for configuring delivery of electrostimulation to specific tissue of a patient, the system comprising a receiver module configured to receive brain anatomy data for a patient and lead position data for a lead forming part of an electrostimulation system, the lead position data indicating a location of the lead in the brain of the patient; a voxel definition module configured to define portions of the patient's brain in voxel form as a voxel data structure, the voxel definition module configured to identify a first non-linear response structure in the brain from the brain anatomy data and identify voxels associated with the first non-linear response structure as a first set of non-linear voxels, and to treat other voxels outside of the first non-linear response structure as linear voxels; a structure selection module coupled to a user interface providing a graphical output allowing a user to identify and select structures in the patient's brain as target structures and as avoid structures; an optimizer configured to identify a plurality of candidate therapies by: a) selecting a steering configuration for issuing output current in a fractional manner across a plurality of electrodes, the electrodes receiving a fraction of a total current; b) determining, for each of a plurality of total current amplitudes, at least a first therapy metric for the non-linear voxels using a non-linear function, and a second therapy metric for the linear voxels for the selected steering configuration; and c) selecting a different steering configuration and repeating a) and b) to generate a plurality of therapy candidates each identifying a therapy metric associated with a particular total current amplitude and steering configuration; and a therapy selection module adapted to present the candidate therapies to a user for selection of one or more candidate therapies for testing on a patient.

Additionally or alternatively, the structure selection module is configured to associate each target structure and each avoid structure with a weight to be used in calculating the therapy metrics.

Additionally or alternatively, the first non-linear response structure is a nerve fiber, and a weight associated with the non-linear structure is calculated by determining neural structures to which the nerve fiber connects.

Additionally or alternatively, the structure selection module is configured to associate a background weight with any volume in the patient neural tissue that would be activated by a therapy.

Additionally or alternatively, the optimization module is configured to identify a combination of total current amplitude and highest metric for each steering configuration that is tested, and selects the combinations of steering configuration and total current amplitude having highest metrics as the candidate therapies.

Additionally or alternatively, wherein each of the therapy metrics are calculated using a cost function analysis by: determining a first partial therapy metric associated with the non-linear response structure using a non-linear function; determining a second partial therapy metric associated with the set of linear response voxels using a linear function; and summing the first partial therapy metric and the second partial therapy metric to determine the first therapy metric.

Additionally or alternatively, the optimizer is configured to determine, for each of a plurality of total current amplitudes, at least a first therapy metric for the non-linear voxels using a non-linear function by determining a quantity of voxels in the non-linear response structure are activated in a given steering configuration and at a given total current amplitude, and applying a non-linear function to the quantity. Additionally or alternatively, the non-linear function is a polynomial function. Additionally or alternatively, the non-linear function is a step function. Additionally or alternatively, the non-linear function comprises two or more segments, each segment corresponding to a range of the quantity, and each segment applying a different linear or non-linear function.

Additionally or alternatively, the optimizer is configured to estimate voltage fields within the first non-linear structure to determine whether the non-linear structure would be activated at a given steering configuration and total current amplitude.

Additionally or alternatively, the therapy selection module is configured to generate and display an image to the user to aid in selecting among the candidate therapies, the image indicating a volume of activation of the neural tissue for at least one candidate therapy.

Additionally or alternatively, the system may further comprise a communications circuit configured to communicate a selected therapy candidate to be tested to a pulse generator of the neurostimulation system, the pulse generator being connected to the lead to allow delivery electrical outputs defined by the selected therapy candidate to the patient.

Additionally or alternatively, the first non-linear response structure is a nerve fiber or a bundle of nerve fibers.

Additionally or alternatively, the first non-linear response structure is a nerve fiber located in the internal capsule, and the lead is positioned to deliver therapy to a target in the thalamus.

Another illustrative and non-limiting example takes the form of a method of configuring a neurostimulations system comprising: receiving, in a computing system, brain anatomy data for a patient and lead position data for a lead implanted in the brain of the patient; defining volume portions of the patient's brain as voxels surrounding the lead, including identifying non-linear voxels associated with a first non-linear response structure in the brain, and to treating other voxels outside of the first non-linear response structure as linear voxels; presenting, via a user interface, a graphical output allowing a user to identify and select structures in the patient's brain as target structures and as avoid structures; identifying a plurality of candidate therapies by: a) selecting a steering configuration for simulating issuance of output current in a fractional manner across a plurality of electrodes on the lead, the electrodes each receiving a fraction of a total current; b) calculating, for the selected steering configuration, and for each of a plurality of total current amplitudes, at least a first therapy metric for the non-linear voxels using a non-linear function, and a second therapy metric for the linear voxels, and summing the first therapy metric with the second therapy metric; and c) selecting a different steering configuration and repeating a) and b) to generate a plurality of therapy candidates each identifying a therapy metric associated with a particular total current amplitude and steering configuration; and presenting to a user, via the graphical user interface, the candidate therapies to a user for selection of one or more candidate therapies for testing on a patient.

Additionally or alternatively, the method further comprises associating each target structure and each avoid structure with a weight to be used in calculating the therapy metrics.

Additionally or alternatively, the first non-linear response structure is a nerve fiber, and a weight associated with the first non-linear response structure is calculated by determining neural structures to which the nerve fiber connects.

Additionally or alternatively, the method comprises associating a background weight with any volume in the patient neural tissue that would be activated by a therapy, and using the background weight to determine a background penalty when calculating the therapy metrics.

Additionally or alternatively, the method comprises selecting the candidate therapies by identifying a combination of total current amplitude and highest metric for each steering configuration, and selecting the combinations of steering configuration and total current amplitude having highest metrics as the candidate therapies.

Additionally or alternatively, each of the therapy metrics are calculated using a cost function analysis by: determining a first partial therapy metric associated with the non-linear response structure using a non-linear function; determining a second partial therapy metric associated with the set of linear response voxels using a linear function; and summing the first partial therapy metric and the second partial therapy metric to determine the first therapy metric.

Additionally or alternatively, calculating the first therapy metric includes determining at least a first therapy metric for the non-linear voxels using a non-linear function by determining a quantity of voxels in the non-linear response structure that are activated in a given steering configuration and at a given total current amplitude, and applying a non-linear function to the quantity. Additionally or alternatively, the non-linear function is a polynomial function. Additionally or alternatively, the non-linear function is a step function. Additionally or alternatively, the non-linear function comprises two or more segments, each segment corresponding to a range of the quantity, and each segment applying a different linear or non-linear function.

Additionally or alternatively, calculating the first therapy metric includes estimating voltage fields within the first non-linear structure to determine whether the non-linear structure would be activated at a given steering configuration and total current amplitude. Additionally or alternatively, presenting the candidate therapies to the user comprises generating and displaying an image to the user to aid in selecting among the candidate therapies, the image indicating a volume of activation of the neural tissue for at least one candidate therapy.

Additionally or alternatively, the method comprises communicating to a pulse generator of the neurostimulation system a selected therapy candidate, and commanding the pulse generator to issue a therapy using the selected therapy candidate to the patient. Additionally or alternatively the method includes the pulse generator issuing the therapy. Additionally or alternatively, the first non-linear response structure is a nerve fiber or a bundle of nerve fibers. Additionally or alternatively, the first non-linear response structure is a nerve fiber located in the internal capsule, and the lead is positioned to deliver therapy to a target in the thalamus.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative DBS system implanted in a patient;

FIG. 2 illustrates details of a directional DBS lead;

FIG. 3 shows an illustrative method in block form;

FIG. 4 is an image of a human brain highlighting select structures;

FIG. 5 is a schematic diagram of brain structures and a DBS lead;

FIGS. 6-7 illustrate simulations of DBS stimulation fields and brain structures;

FIG. 8 shows an illustrative method incorporating non-linear and linear structures in analysis of DBS therapies;

FIGS. 9A-9D illustrate simulations of DBS stimulation fields;

FIGS. 10A-10E show various mathematical functions for estimating stimulation field effects; and

FIG. 11 shows an illustrative DBS system.

### DETAILED DESCRIPTION

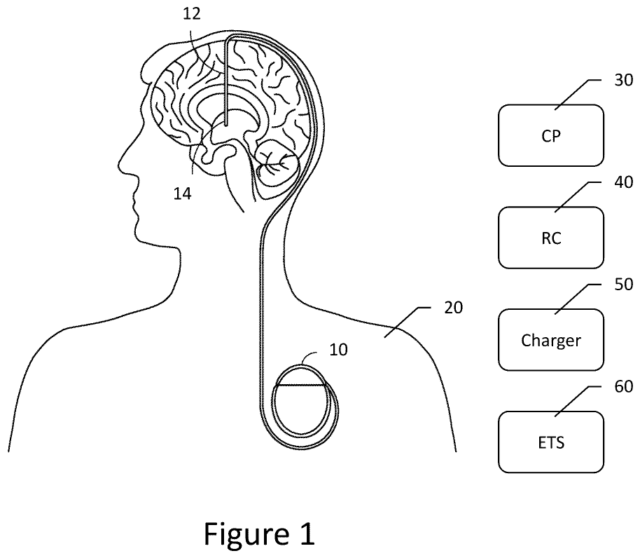

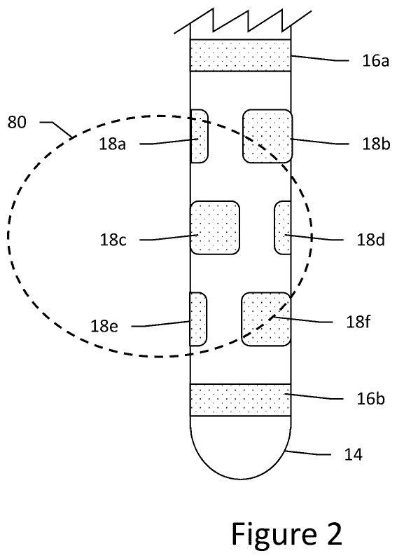

FIG. 1 shows an illustrative DBS system implanted in a patient. The system comprises a pulse generator 10, shown implanted in the pectoral region of a patient 20. The pulse generator 10 is coupled to a lead 12 which extends subcutaneously to the head of the patient 20, through a burr hole formed in the patient's skull, and then into the brain of the patient. In the example shown, the lead 12 includes a plurality of electrodes positioned near the distal end 14 of the lead, such as shown below in FIG. 2. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulata, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Target” regions as used herein are brain structures associated with therapeutic benefits, and avoidance regions or “Avoid” regions are brain structures associated with side effects.

Conditions to be treated may include dementia, Alzheimer's disease, Parkinson's disease, dyskinesias, tremors, depression, anxiety or other mood disorders, sleep related conditions, etc. Therapeutic benefits may include, for example, and without limitation, improved cognition, alertness, and/or memory, enhanced mood or sleep, avoidance of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of FIG. 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 30 may be, for example, a laptop or tablet computer and can be used by a physician, or at the direction of a physician, to obtain data from and provide instructions the pulse generator 10 via suitable communications protocols such as Bluetooth or MedRadio or other wireless communications protocols, and/or via other modalities such as inductive telemetry.

A patient remote control (RC) 40 can be used by the patient to perform various actions relative to the pulse generator 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 40 can communicate via similar telemetry as the CP 30 to control and/or obtain data from the pulse generator 10. The patient RC 40 may also be programmable on its own, or may communicate or be linked with the CP 30.

A charger 50 may be provided to the patient to allow the patient to recharge the pulse generator 10, if the pulse generator 10 is rechargeable. Some pulse generators 10 are not rechargeable, and so the charger 50 may be omitted. The charger 50 can operate, for example, by generating a varying magnetic field to activate an inductor associated with the pulse generator 10 to provide power to recharge the pulse generator battery, using known methods and circuitry.

Some systems may include an external test stimulator (ETS) 60. The ETS 60 can be used to test therapy programs after the lead 12 has been implanted in the patient to determine whether therapy will or can work for the patient 20. For example, an initial implantation of the lead 12 can take place using, for example, a stereotactic guidance system, with the pulse generator 10 temporarily left out. The lead 12 may have a proximal end thereof connected to an intermediate connector (sometimes called an operating room cable) that couples to the ETS 60. After lead 12 has been implanted and coupled to the ETS 60, the ETS can be programmed using the CP 30 with various therapy programs and stimulation parameters. Once therapy suitability for the patient is established, the permanent pulse generator 10 is implanted and the lead 12 is connected thereto, with the ETS then being removed from use.

The pulse generator 10 may include operational circuitry for generating output stimulation programs and/or pulses in accordance with stored instructions. Some examples of prior versions of such circuitry, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional background and/or examples of the pulse generator 10, CP 30, RC 40, Charger 50, and ETS 60 can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

FIG. 2 illustrates details of a directional DBS lead. The distal end 14 is shown, and a plurality of electrodes are shown as well. Two ring electrodes 16a, 16b (collectively ring electrodes 16) can be provided as shown, and a number of segmented electrodes are shown at 18a, 18b, 18c, 18d, 18e, 18f (collectively, segmented electrodes 18). Each electrode 16, 18 may be separately addressable in the system, such as by using a pulse generator having multiple independent current control (MICC) or multiple voltage sources.

MICC is a stimulus control system that provides a plurality of independently generated output currents that may each have an independent quantity of current. The use of MICC can allow spatially selective fields to be generated during therapy outputs. The term “fractionalization” may refer to how the total current issued by the pulse generator via the electrodes is divided up amongst the electrodes 16, 18 on the lead. It should be noted that the pulse generator canister may serve as an indifferent electrode or as a return electrode for therapy outputs. Alternatively, one of the lead electrodes (such as a ring electrode 16 or one or more of the segmented electrodes 18) may instead be used as a return electrode. Thus, for example, the electrodes 16, 18 on the lead may serve as cathodes while pulse generator canister serves as an anode during one phase of stimulation pulse delivery. In another example, some of the lead electrodes 16, 18 serve as cathodes, while other lead electrodes 16, 18 serve as anodes during one phase of stimulation pulse delivery.

Examples of electrical leads with segmented or directional lead structures are shown, for example and without limitation, in US PG Pat. Pubs. 20100268298, 20110005069, 20110078900, 20110130803, 20110130816, 20110130817, 20110130818, 20110238129, 20110313500, 20120016378, 20120046710, 20120071949, 20120165911, 20120197375, 20120203316, 20120203320, 20120203321, 20130197602, 20130261684, 20130325091, 20130317587, 20140039587, 20140353001, 20140358207, 20140358209, 20140358210, 20150018915, 20150021817, 20150045864, 20150021817, 20150066120, 20130197424, and 20150151113, and U.S. Pat. Nos. 8,483,237 and 8,321,025, the disclosures of which are incorporated herein by reference.

For example, a directional lead as shown in FIG. 2 may be used to generate a stimulation field as illustrated at 80 in FIG. 2. The outer boundary of field 80 may be understood as representing an equipotential or equal field boundary, within which the electrical field is higher than an activation threshold, and outside of which the electrical field is below the threshold, for purposes of illustration. An activation threshold may represent or approximate a voltage/field threshold at which neural cells will activate. Activation thresholds may be determined on a population basis, such as by relating to a voltage/field at which a 50% likelihood of activation of 50% of the cell population is determined, thought other boundaries/thresholds can be used. The shape of the field can be adjusted, as described variously in the references incorporated by reference above, by modifying the fractionalization of current issued via the electrodes. An electrical field as shown at 80 may be (roughly) generated by using electrode 18c as a cathode, and surrounding electrodes 18a, 18e, and 18d as anodes, for example. The actual characteristics of fractionalization may be more sophisticated than this simple example.

A related concept to the field shown at 80 in FIG. 2 is that of stimulation field modeling (SFM). In SFM, the tissue is modeled, for example, using finite element models in which the lead body is treated as an insulator, surrounded by a thin encapsulation sheath, and surrounded by neural tissue. The neural tissue may be modeled as isotropic and homogenous, though more sophisticated modelling can also be used if desired. A set of model voxels are defined around the lead, breaking up the volume into small segments, each of which can be analyzed within the model. The outer boundaries of the SFM can be determined using a population-based activation threshold as described above. The result can be that at a given fractionalization and total stimulation current, an SFM can be generated as a three-dimensional surface surrounding a portion of the lead and encompassing a volume of neural tissue. Field 80 may be, for example, understood as a two-dimensional representation of a slice of the SFM. The SFM can be used as a visual tool for illustrating, to a patient or physician, what tissue is or is not being stimulated by a given fractionalization and total current.

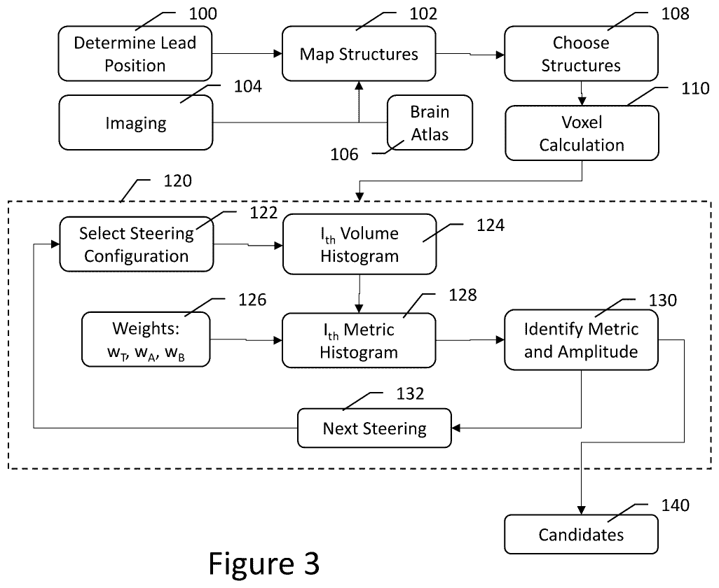

FIG. 3 shows an illustrative method in block form. In the illustrative method, lead position in the patient is determined at 100. For purposes of this illustration, lead position in the brain will be explained, however, the present invention may be used when modeling and optimizing neural stimulation in other parts of the body. The lead position 100 may be determined by use of imaging modalities such as X-ray, CT scan, MRI, or others, after the lead has been implanted.

Next, the system maps structures, as indicated at 102. Mapping structures 102 may include the use of pre-operative and/or post-operative imaging 104 and data from a brain atlas 106 to identify structures within the brain. A brain atlas 106 may include data from a population of patients indicating the general position and nature of structures in the brain, allowing the images to be referenced against population examples. Illustrative structures may include the thalamus, the globus pallidus, the subthalamic nucleus, and/or other structures listed above.

The user or physician then chooses structures at block 108. Structures may be identified as target structures or avoid structures. Target structures are those that the physician determines should be stimulated as much as possible, and avoid structures are those that the physician determines should not be stimulated, to the extent possible. Typically, target structures are associated with therapy benefits, and avoid structures are those that are associated with side effects.

Voxel calculation occurs as indicated at 110. The voxel calculation 110 defines a grid of voxels in the tissue region around the brain, and determines characteristics of tissue in each voxel, as further described below. As used herein, “voxel” refers to any segment of volume used in an analysis, regardless of shape and may include cubes, spheres, partial cylinders, partial toroidal shapes, etc. For simplicity, the figures show shapes defined as cubes (as voxels may be defined in Cartesian coordinates), and may reference any of world, anatomical or image coordinate systems. Other voxel definitions and coordinate systems (such as spherical or polar coordinates) can be used if desired. Some systems may use an anatomical reference for the relevant coordinate system to define both lead position and structure positions/locations. The skilled person will understand transformations from one coordinate system to another. The voxel calculation 110 includes identification of which voxels contain target and avoid structures, using the choices made at block 108.

An optimization follows in an iterative block 120. The structure choices 108 and voxel calculation 110, and/or device history or other inputs, are used to determine an initial steering configuration 122, which is then used to determine an Iₜₕ table. The contents of the Iₜₕ table indicate, for each voxel using a given steering state and fractionalization, the minimum total current that would be needed to trigger neural activity in the given voxel. The Iₜₕ table may be sorted from least to greatest Iₜₕ. The values of the Iₜₕ table at the non-zero voxel locations are used to create Iₜₕ Volume Histograms 124, specifying the change in stimulated volume of each structure at each of a range of amplitudes. That is, each voxel is characterized as activated or not activated at a plurality of amplitudes, and each of the volume histograms 124 identifies which voxels are or are not activated at a given amplitude using the steering configuration under analysis.

The contents of these Iₜₕ Volume Histograms 124 are combined with weights 126, to generate the Iₜₕ Metric Histogram 128. In a simple approach, there may be two user-adjustable weights, and one pre-set weight: target volume weight, w_{T} may be pre-set to 1, avoid structure weight, w_{A}, and background weight w_{B} may be user adjustable; other approaches to weighting for target, avoid and background structures may be used. The metric histogram 128 thus indicates, for each of a plurality of amplitudes, the resultant metric in which voxels that are activated are weighted in accordance with weights 126.

As noted, the product of the weighting values 126 of each voxel and the Iₜₕ Volume Histogram 124 is referred to as an Iₜₕ Metric Histogram 128. The per-amplitude change in metric values are integrated to determine the highest metric value and the current amplitude that generates the highest metric value, at block 130. These values, and those generated by previous iterations through the optimization, are used to generate a next steering state, prompting the next iteration, as indicated at 132. If an exit condition is met, such as by showing that the metric is not increasing with new steering configurations, the iterations in 120 terminate and the resulting candidates, including the various maximum metrics and amplitudes, are analyzed at 140.

During the analysis in FIG. 3, each voxel may be understood as having its own value or metric, depending on whether the voxel is in a target region, avoid region, or background (neither target nor avoid). At a high level, the total metric may be understood as indicated at Equation 1:

$$metric = \sum ( {v}_{target} - ( {v}_{avoid} * {w}_{A} ) - ( {v}_{s f m} * {w}_{B} ) ) \qquad Eq . l$$

Where v_{target} is the volume of stimulated target tissue, v_{avoid} is the volume of the stimulated avoid region, and v_{sfm} is the total stimulated region at the optimized amplitude. In Equation 1, w_{A}, and w_{B} are as previously described, and each may be user adjustable; if desired, a target weight, w_{T}, (set to 1 and omitted in Equation 1) may be included and would multiply with v_{target}. An analogous version of Equation 1 may also be used on a voxel by voxel basis to populate the metric histogram 128. This general form is modified further in several examples below.

Further examples and possible details for use in the preceding description of the algorithm in FIG. 3 may be found in U.S. Pat. No. 11,195,609, the disclosure of which is incorporated herein by reference for details of a voxelization, histogram, and optimization procedure. However, the preceding description is one simply way that optimization 120 can be implemented. In other examples, a different order of operations may be used. For example, in an alternative, with each given steering configuration, a seeking algorithm may be used to test different amplitude measures without generating a histogram at all, with the seeking algorithm used through several iterations until a current amplitude at a given steering configuration is determined that maximizes the metric as calculated above in Equation 1.

While a simplest approach to finding highest metric values may be to sweep all available steering and amplitude configurations (along with other parameters such as pulse width, shape, frequency, etc.), the computational burden of such a procedure may be excessive. It may be preferable to use a more selective approach. In some examples, for a given patient's anatomy, lead position, and medical condition, the optimization at 120 may use similarity analysis to identify similar patient characteristics in a database, and may use such analysis to generate starting points for the optimization 120. As noted, these are merely examples.

Those combinations of steering configurations and stimulation parameters that yield highest metrics may be characterized as candidate therapies, as indicated at 140. Candidate therapies 140 may further be rated or analyzed using secondary factors, such as power consumption. The candidate therapies may be presented to the physician. The physician can then select steering configurations and stimulation parameters for use in subsequent testing of the system. Testing can occur using an ETS or implantable pulse generator, as desired.

In prior systems, the weighting schemes and indications of target and avoid structures deal in linear or continuous inputs and outputs. For example, when considering Equation 1, above, the cost function only uses a linear weighting system, in which the cost or benefit associated with each term is calculated by multiplying the volume of activation by a constant. This is not reflective of certain neural structures. For example, nerve fibers, once activated by an electrical output, will not further respond to increases in the volume of such fibers that are activated. Once a threshold activation of the fiber occurs, additional stimulation of the fiber will not have a further effect. Methods and systems that enhance Equation 1 to account for non-linear response are desired.

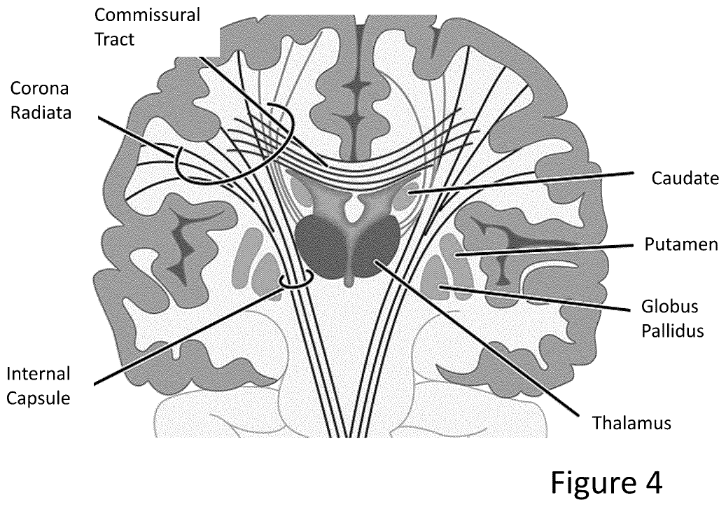

FIG. 4 is an image of a human brain highlighting select structures. The simplified image illustrates positions of structures including the caudate, putamen, globus pallidus, and thalamus. Fiber tracts are also shown, including the internal capsule, which has nerve fibers that contribute to the corona radiata, and the commissural tract. These nerve fibers carry signals between structures in the brain. As a result, stimulation of one part of a fiber tract can have the effect of sending a neural signal along a nerve fiber to other parts of the brain, including to connected structures at which nerve fibers terminate. For example, stimulation of a fiber tract can cause signaling to pass in both afferent and efferent directions along a nerve fiber located in the fiber tract, potentially activating structures at either end of the nerve fiber. As may also be observed, commissural fiber tracts as well as the internal capsule pass alongside structures that may be the targets of DBS. In particular, therapy directed to the thalamus will necessarily be issued in the vicinity of the internal capsule.

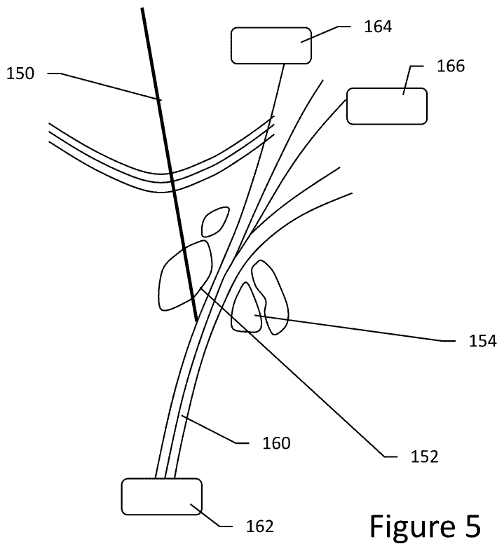

FIG. 5 is a schematic diagram of brain structures and a DBS lead. Here, a lead 150 has been implanted with a distal end near and/or alongside the thalamus 152. The globus pallidus is shown at 154, and the internal capsule 160 can be seen in this same region. The nerve fibers inside the internal capsule 160 can be seen to reach additional structures 162, 164, 166 in both afferent and efferent directions. For example, afferent fibers of the internal capsule may pass from cell bodies of the thalamus to the cortex, and efferent fibers of the internal capsule may pass from cell bodies of the cortex to the cerebral peduncle of the midbrain. Nerve fibers in the internal capsule contribute to the corona radiata. The lead 150 is therefore positioned in a location where some effects of therapy fields can be approximated or measured using a linear response model (such as shown in Equation 1), while other effects can have very different functions, better modeled as step functions and/or with quadratic or other functions.

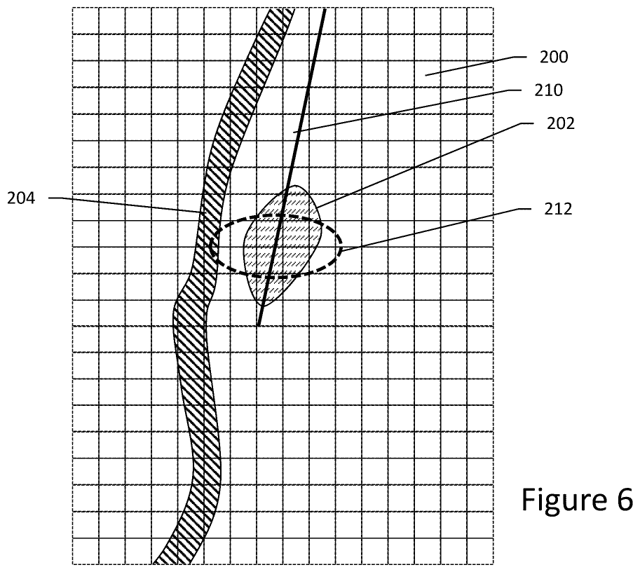

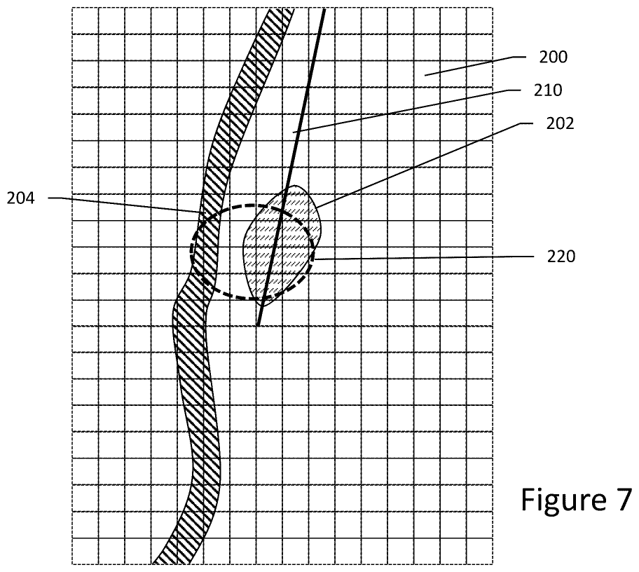

FIGS. 6-7 illustrate simulations of DBS stimulation fields and brain structures. In these Figures, a grid pattern is shown to represent voxels; it should be understood that the two-dimensional representation would have a thickness as well so that each box represents a three-dimensional structure, which may be a cube for example. In FIG. 6, the grid is shown at 200. A neural structure is shown at 202 and may take the form of, for example, the thalamus. A fiber tract 204 is shown as well; the fiber tract 204 may be, for example, the internal capsule, containing a number of nerve fibers. A lead 210 is shown with a distal end thereof passing alongside the neural structure 202. An SFM 212 is shown illustratively, based on a hypothetical combination of steering (fractionalization) and amplitude parameters. As can be seen, a portion of the SFM 212 contains a volume of the neural structure 202, but also interacts with the fiber tract 204.

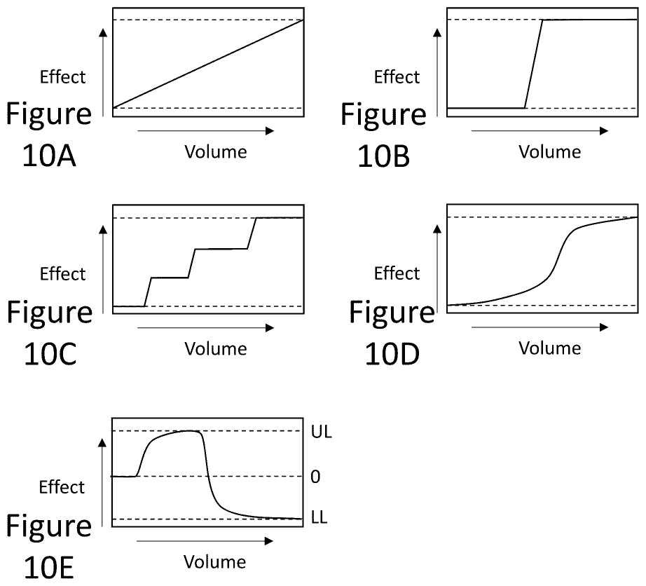

The volume of the neural structure 202 that is encompassed by the SFM may have a therapeutic effect (side effect or benefit) that is generally linear with respect to the volume within the SFM. That is, a linear formula as used in Equation 1, and/or a graph as shown in FIG. 10a, below, may adequately characterize the relationship between stimulated volume and therapeutic effect. The neural structure 202 may be considered a linear response structure in some examples.

The volume of the fiber tract 204 that is encompassed may include nerve fibers, each of which may has a therapeutic effect (side effect or benefit) that is non-linear or even non-continuous with respect to the stimulated or activated volume. For example, a step function, a multi-step function, a polynomial function, and/or a segmented response, as illustrated in FIGS. 10B-10E, below, may better characterize the relationship between stimulated volume and therapeutic effect. The nerve fibers in the fiber tract (or any other neural structure that is not a linear response structure) may be considered a non-linear response structure in some examples.

In some examples, the fiber tract 204 and the nerve fibers therein are, collectively, an avoid structure, either directly as stimulation of the nerve fibers in fiber tract 204 may be undesirable, or because stimulation of the nerve fibers in fiber tract 204 is likely to cause afferent or efferent neural activity at the structures to which the fiber tract 204 connects, and those further activities may cause known or unknown side effects. In other examples, fiber tract 204 may be a target structure, either directly because stimulation of the fiber tract 204 may be desirable, such as to help stimulate the cells thereof to maintain vitality or enhance or maintain function of the fiber tract, or because stimulation of the nerve fibers in the fiber tract 204 may have afferent or efferent benefits by causing activation of remote structures to which the nerve fibers of fiber tract 204 are connected.

FIG. 7 is similar to FIG. 6, but illustrates a different therapy configuration, with one or more steering/fractionalization and/or amplitude parameters modified. Here, the SFM 220 is less oval and more circular, encompassing more of the neural structure 202, but also more of the fiber tract 204. Comparing FIG. 6 to FIG. 7, the nonlinear response of nerve fibers in the fiber tract 204 means that the therapeutic effect (beneficial or otherwise) is not adequately characterized by a linear equation. For example, depending on characteristics of the fiber tract 204, the beneficial effects, or side effects, caused by stimulation of the nerve fibers in fiber tract 204 in FIG. 7 may be the same as the effects caused by stimulation as shown in FIG. 6. Alternatively, the lesser volume in the SFM in FIG. 6 may mean that the nerve fibers in fiber tract 204 experience no effect due to failure to activate a minimum threshold volume, while the larger volume in FIG. 7 may have a maximum effect including both stimulation of the nerve fibers in fiber tract 204 but also connected efferent and/or afferent structures elsewhere in the brain or body.

In illustrative examples, brain atlas or other information, such as a library of therapeutic data captured from a patient population, may be used to determine an appropriate model to use for the effect of stimulation on a non-linear response structure. Some illustrative non-linear functions are illustrated below in FIGS. 10B-10E. A most basic approach is simply to consider the fiber activated if it passes through the SFM, or if a portion resides in the SFM.

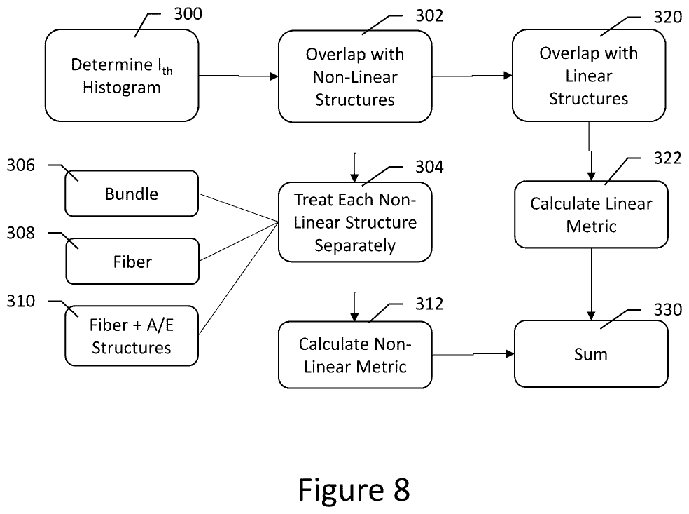

FIG. 8 shows an illustrative method incorporating non-linear and linear structures in analysis of DBS therapies. At block 300, a threshold current (Iₜₕ) histogram is generated. The Iₜₕ histogram may be derived from a given steering configuration/fractionalization, as above. An overlap of the Iₜₕ histogram with non-linear structures is then determined at 302. Each non-linear structure can then be treated individually at 304. As noted, non-linear structures may include bundles of fibers 306, fibers individually 308, or fibers plus any afferent or efferent structures 310. In other examples, non-linear structures may include any neural structure that is modeled having a non-linear response to stimulation. In an illustrative example, each voxel may be binned by the structure to which it belongs, such as by linear, nonlinear, and/or background bins, for example. Multiple bins may exist for each of non-linear and linear categories. Each bin may be associated with a memory address indicating the specific function to be used in analyzing the nature of voxel stimulation for purposes of metric determination.

A non-linear metric is then calculated at 312, as the sum of a metric calculated for each of the non-linear structure bins. In an illustrative example, the following formula may be used at block 312 to calculate the non-linear metric (M_{NL}):

$${M}_{N L} = {\sum }_{∀ k} {f}_{k} ( {v}_{k} ) \qquad \{ Equation 2 \}$$

Where each k represents a non-linear structure, fₖ is the nonlinear function for the k^{th} nonlinear structure, and vₖ is the quantity of voxels or percentage fill of the k^{th} nonlinear structure by the Iₜₕ histogram. A plurality of non-linear metrics may be calculated using a plurality of amplitudes for the total current, similar to the generation of the Iₜₕ Metric Histogram at 128 in FIG. 3.

Illustrative nonlinear functions may include:
Exponential functions such as f(v)=A*exp(v) . . .
Polynomial functions such as f(v)=A*v+B*v²
Rational functions, which may combine exponential and/or polynomial functions
Segmented functions, such as:
   f(v)=
      Function f1, if v is less than a first threshold
      Function f2, if v is greater than or equal to the first threshold and less than a second threshold, or
      Function f3 if v is greater than or equal to the second threshold
   Where f1, f2, and f3 may be constants, linear functions, or non-linear functions including exponential or polynomial functions, and the thresholds may be selected as desired, such as from 0 to a maximum number of voxels possible in the non-linear tissue volume, if voxel count is used, or 0% to 100% if percentage fill is considered.
      The above functions are simplified, and additional terms may be included. Some segmented functions may be approximated by the use of exponential, polynomial and/or rational functions, as desired. A basic segmented function may be as follows:
f(v)=0, if v<vₘᵢₙ; else 1
   Where vₘᵢₙ is a minimum fiber tract interaction with the Iₜₕ histogram, above which the fiber is considered maximally activated.

Each of the individual fₖ functions may be associated with a predetermined or user configurable weight, as desired, and may generate positive or negative metric values. The weight may be positive or negative, as desired. For example, positive metric values may indicate stimulation of target structure(s), and negative metric values may indicate stimulation of avoid structure(s). Further, as to configurable weight, each non-linear function may have a weight set to account for any effect on afferent or efferent structures associated with a given fiber tract or bundle, as desired.

Returning briefly to FIG. 7, the histograms noted in FIG. 8 may be understood as having a plurality of categories. One histogram may be associated with structure 204, and may include all of the voxels having at least a predetermined fraction thereof filled by structure 204. For example, a histogram, H₂₀₄, for structure 204 may identify all voxels having at least 50% of the volume thereof (or some other percentage from 1% to 100%, inclusive) occupied by structure 204. Another histogram, H₂₀₂, may identify voxels for structure 202. The sets of associated voxels for each histogram can then be compared against the set of voxels in the Iₜₕ histogram.

In some examples, each individual structure in the voxel space may be analyzed separately, including both nonlinear and linear response structures. Alternatively, as shown in FIG. 8, the linear structures may be analyzed together by finding overlap at 320 and calculating the linear metric at 322 using, for example, Equation 1. Finally, the results of the analyses at 312 and 322 can be summed together at 330, providing a final metric for the simulated therapy. The process can be repeated in an iteration block as shown above at 120 in FIG. 3, to eventually yield a set of candidate therapies for block 140.

In some further examples, the idea of probabilistic structures may be introduced as well. For example, each histogram analysis may include volume details relating individual neural structures. A probabilistic structure can be generated from imaging data and brain atlas inputs, if desired, and indicates using a plurality of probability shells the likely location of a given structure. For example, a structure such as the internal capsule is bounded by the capsule sheath, and the precise location that the sheath starts is not knowable based on imaging and atlas data. A series of probability shells can indicate location while acknowledging uncertainty. For example, a given voxel may be entirely within a 10% probability shell, and half inside a 20% probability shell. The probability that the neural structure (or a portion thereof) is in the given voxel can be estimated at 15% (10% times half the shell volume, for 5%, plus 20% times half the shell volume for 10%, summed to 15%). Probability shell analysis can be used to discount the likelihood that a given voxel which is inside the SFM boundary and also within a probability shell for a neural structure actually contains the neural structure. Probability shell analysis can be used, in some examples, to discount the volume of activation for a given structure/histogram, for example.

In still another example, fiber structures may be separated out for analysis different from the rest of the neural tissue. Here, rather than analyzing whether the fiber will be activated according to a general Iₜₕ analysis, in which each voxel is individually analyzed using a population-based activation threshold, each point in the fiber may be identified as part of a continuous structure. Then, along the spatial coordinates of the fiber, the voltage field value at each voxel/point can be gathered, to determine the total current level at which the fiber would activate. In doing so, the non-linear structures may be omitted entirely from the linear structure analysis, or may be treated as background in the linear structure analysis, with separate analysis creating additional positive or negative metric values from the analysis of nerve fibers.

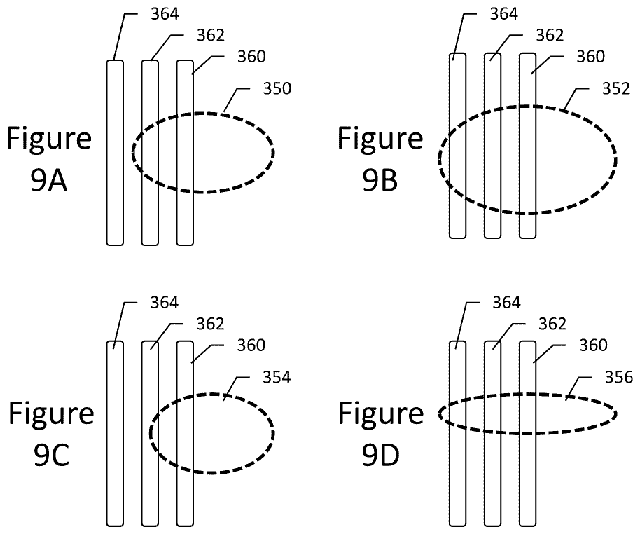

FIGS. 9A-9D illustrate simulations of DBS stimulation fields, and the effect on individual fibers. In FIG. 9A, an SFM is shown at 350 interacting with a first fiber 360 as well as a second fiber 362. A third fiber 364 is outside the SFM. Depending on the nature of each fiber and the responsiveness of each fiber to stimulation, the set of fibers 360, 362, 364 may be each treated individually and may have very different responses. For example, fiber 360 may be an efferent fiber carrying sensory information, fiber 362 may be an afferent fiber carrying motor commands, and fiber 364 may be an efferent fiber carrying additional sensory information. In an example, fiber 360 may be treated as fully activated by the SFM 350, while fibers 362 and 364 may be treated as unaffected.

Continuing the example, a different amplitude setting may be used at FIG. 9B to generate a larger SFM 352. The effect on fiber 360, however, is the same for FIG. 9A as for FIG. 9B, since fiber 360 was already fully activated by the first SFM 350, so no further penalty or benefit is calculated for fiber 360. Fiber 362, however, has a larger area now activated, and may now be treated as fully activated, and Fiber 364 may also now be treated as fully activated for FIG. 9B.

Reducing amplitude from FIG. 9A to FIG. 9C may instead reduce the size of the SFM, as indicated at 354. In this example, the effects on each of fibers 360, 362 and 364 are the same for FIG. 9C as for FIG. 9A, meaning that the metric score is unchanged from FIG. 9A to FIG. 9C.

Modifying the steering or fractionalization settings may result in a different SFM shape as shown in FIG. 9D. Here, the three fibers 360, 362, 364 are each in the SFM, but each may respond differently. By treating each histogram separately, separate functions can be applied to model stimulation effects. In the example, continuing the above discussion, the volume of activation of fibers 360 and 364 necessary to fully stimulate each fiber may be relatively lower than it is for fiber 362. This may occur if, for example, fiber 362 is of a different type or size than the other fibers 360, 364. As a result, FIG. 9D may indicate activation of each of fibers 360, 364, but not fiber 362, by the application of separate histograms and non-linear functions to each fiber.

FIGS. 10A-10E show various mathematical functions for estimating stimulation field effects, FIG. 10A shows a linear function, in which the clinical effect or stimulation field effect changes in a direct, proportional relationship with the increasing volume of the relevant neural tissue structure within the SFM or Iₜₕ histogram. At FIG. 10B, a step-like function is shown, in which the therapeutic effect (which may be benefit or side effect/harm) is seen to be very small, or even zero, until the volume of the relevant neural tissue structure subject to stimulation crosses a boundary, at which point the effect can be seen to jump. FIG. 10C shows another step function, here with multiple steps. For example, if a fiber tract or bundle connects to multiple structures, there may be multiple steps in the effects function. FIG. 10D shows an example in which a polynomial function may be used. Using a polynomial may reduce the need to refer to look-up tables, as may occur with step-wise functions of FIGS. 10B-10C, allowing faster calculations with reduced memory requirements. FIG. 10E shows another illustration. Here the function can result in a positive effect or a negative effect, as the volume of the relevant neural tissue structure increases. An example for FIG. 10E may be one in which stimulus is desirable to help maintain vitality of a neural structure such as a fiber tract that may be subject to degradation or cell death, however, if too much stimulation occurs, side effects may include activation of neural structure connected to the target fiber tract, causing side effects that negate the clinical benefit. The function as shown may be implemented as a polynomial and/or a rational polynomial, if desired. Determination of which function may apply to a given structure, such as a nerve fiber, can be based on patient specific information (such as patient history), analysis of structures, or data from other patients having similar systems, symptoms or anatomy, for example, or from any other suitable source.

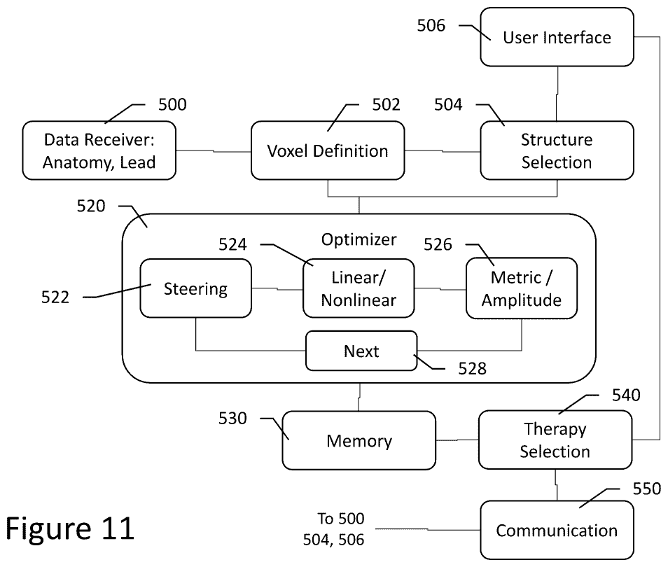

FIG. 11 shows an illustrative DBS system. The system may include a data receiver block 500. For example, a communication circuit 550 may be coupled to a software or hardware module that is configured to receive and store information related to the patient anatomy and lead position. The data in block 500 is then used for voxel definition at 502, which may be implemented as another software module and/or by a separate or dedicated circuit (application specific integrated circuit, microcontroller, etc.). The voxel definition and anatomy data from block 500 may include identification or definition of different neural structures as having linear or non-linear responses, setting up the system to use the above methods for analysis of metrics for side effects and benefits of therapy. The voxel definition block 502 provides a voxelization to a structure selection block 504.

The structure selection block 504 is configured to receive user/physician inputs from a user interface 506, which may include one or more of a keyboard, mouse, trackball, touchscreen, monitor/output screen, voice or other audio input/output devices, etc. The user interface 506 allows the user/physician to identify and select target and avoid regions in the patient anatomy. Additionally, in some examples, the user interface 506 allows the user/physician to designate which neural structures are linear response structures and/or non-linear response structures. The user interface 506 may be used to allow the user/physician to select or modify the background and avoid structure weights that are applied in the functions used to analyze neural structure, the Iₜₕ histogram and metrics. The physician/user may determine the functions to use for particular nonlinear response structures if desired, though in some examples these functions are defined in advance with data input to the data receiver at 500, such as based on neural anatomy knowledge for a given population. Functions may also be generated using individualized testing in a given patient, if desired.

Structure selection may be applied to the voxel definitions to identify, on a voxel-by-voxel basis, the value of each target and avoid voxel, as well as those voxels that are neither target nor avoid. The voxel definitions from 502 and target and avoid structure data from 504 are passed to the optimizer block 520. In some examples, the data going to block 520 identifies voxels in which various neural structures reside or are likely to reside. These may include each of nonlinear and linear structures, as well as weights and other data that can be used to generate metrics related to therapy benefit, side effects, etc.

The optimizer 520 may perform the steps illustrated above in FIGS. 3 and 8. For example, a plurality of iterations are performed. At a given steering or fractionalization 522, linear and non-linear activation is analyzed and weight metrics are applied thereto using a range of potential current amplitudes at 524. The result is a set of metrics and associated amplitudes 526 associated with the fractionalization or steering configuration. A next steering configuration or fractionalization is selected 528, and the next iteration is executed. Throughout the process, data pairing metrics, amplitudes and steering configurations is stored in memory 530.

The metrics and amplitudes block 526 may include a cost function analysis module that determines partial metrics for each of the identified non-linear structures, as well as the linear structures. For example, the positive (therapy benefit) and negative (side effect or background) weights discussed previously are applied to each voxel or volume segment within each of the identified structures, generating the partial metrics, which are then summed together to obtain a final metric at 330 for the given steering configuration and current amplitude. A plurality of current amplitudes, and resultant metrics, can be analyzed together before shifting the analysis to a subsequent steering configuration.

When an exit condition is met, such as exhaustion of potential steering configurations, or a determination that further analysis is not going to result in better metrics, block 520 stops iterating. A therapy selection block 540 obtains the “best” metrics from memory 530 and uses these as candidate therapies. SFMs can be determined using the steering and amplitude metrics for the candidate therapies. The SFMs may aid in physician or user understanding of the spatial characteristics of a proposed or candidate therapy.

The candidate therapies are then presented by a therapy selection module 540 to the user/physician to do one or more of approving a proposed therapy, or selecting among several highest scoring therapies. Once a user has selected a therapy for implementation, a communications block 550 is used to communicate the therapy to a pulse generator or ETS. The pulse generator or ETS then delivers the selected therapy to the patient.

It may be noted that a therapy which is optimized in this manner may be used with additional limitations placed on the patient remote control relative to existing systems. For example, modifying amplitude would change the SFM and may adversely affect resultant metrics, including the amount of background and avoid region stimulation. Thus, the therapy program may be provided to the pulse generator or ETS with a limitation in place preventing the patient RC from being used to adjust therapy amplitude, for example, in ways that would potentially cause greater effects to the metric than might otherwise be expected by, for example, causing a nerve fiber to activate and creating a non-linear response.

Block 528 may use an artificial intelligence or other approach, including regression analysis, to yield a seeking function that efficiently identifies steering and fractionalization sequences to be tried in each iteration. Block 528 may be configured to receive structure selection data that can inform the process of choosing steering configurations, in an example. For example, the AI may have stored therein a database of steering selection configurations and approximate field models, (such as determined using preset amplitude, pulsewidth or other data). By generically comparing the database of steering configurations to received structure selections, the AI may be able to eliminate a large portion of possible steering configurations quickly, to construct a shortened list. Some illustrative methods include iterative optimization methods such as gradient descent search, genetic algorithms, simulated annealing, random coordinate descent, particle swarm, fuzzy logic, among other search algorithms. Various details of the searching process are explained as well in U.S. Pat. No. 11,195,609, the disclosure of which is incorporated herein by reference.

While much of the above discussion focuses on use for DBS, other tissue regions can also be treated. For example, anatomical mapping may be used to identify neural and/or other structures to be targeted or avoided during other therapy, such as spinal cord stimulation (SCS), peripheral nerve stimulation, occipital nerve stimulation, muscles and muscle nerve fibers, therapies directed to the digestive tract, or other regions, and Vagus nerve stimulation. As an example, in spinal cord stimulation, the spinal cord carries neural signals to and from various different parts of the body. With knowledge as to which parts of the spinal cord at a given vertebral level carry signals to and from what part of the body. With this knowledge, therapy and avoid regions in the spinal cord may be defined/determined. If a plurality of leads are present, or a paddle lead, at the given vertebral level, a steering and Iₜₕ analysis, including linear and/or non-linear structures, may be determined for the given vertebral level using a spinal cord atlas as well as lead implantation/position data (such as from an X-ray or other imaging system). Patient specific data, such as from testing, mapping dermatomes, etc., may also be used. A similar process as just described may be used to define therapy that targets the portion of the spinal cord carrying neural signals (such as pain signaling) that are to be interfered with, while other portions (such as carrying motor signals or affecting non-target regions) are to be avoided. The metric calculations described above can then be used to optimize steering to limit side effects and achieve desired therapy.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A system for configuring delivery of electrostimulation to specific tissue of a patient, the system comprising:
a receiver module configured to receive brain anatomy data for a patient and lead position data for a lead forming part of an electrostimulation system, the lead position data indicating a location of the lead in the brain of the patient;
a voxel definition module configured to define portions of the patient's brain in voxel form as a voxel data structure, the voxel definition module configured to identify a first non-linear response structure in the brain from the brain anatomy data and identify voxels associated with the first non-linear response structure as a first set of non-linear voxels, and to treat other voxels outside of the first non-linear response structure as linear voxels;
a structure selection module coupled to a user interface providing a graphical output allowing a user to identify and select structures in the patient's brain as target structures and as avoid structures;
an optimizer configured to identify a plurality of candidate therapies by:
a) selecting a steering configuration for issuing output current in a fractional manner across a plurality of electrodes, the electrodes receiving a fraction of a total current;
b) determining, for each of a plurality of total current amplitudes, at least a first therapy metric for the non-linear voxels using a non-linear function, and a second therapy metric for the linear voxels for the selected steering configuration; and
c) selecting a different steering configuration and repeating a) and b) to generate a plurality of therapy candidates each identifying a therapy metric associated with a particular total current amplitude and steering configuration; and
a therapy selection module adapted to present the candidate therapies to a user for selection of one or more candidate therapies for testing on a patient.

2. The system of claim 1, wherein the structure selection module is configured to associate each target structure and each avoid structure with a weight to be used in calculating the therapy metrics.

3. The system of claim 2, wherein the first non-linear response structure is a nerve fiber, and a weight associated with the non-linear structure is calculated by determining neural structures to which the nerve fiber connects.

4. The system of claim 1, wherein the structure selection module is configured to associate a background weight with any volume in the patient neural tissue that would be activated by a therapy.

5. The system of claim 1, wherein the optimization module is configured to identify a combination of total current amplitude and highest metric for each steering configuration that is tested, and selects the combinations of steering configuration and total current amplitude having highest metrics as the candidate therapies.

6. The system of claim 1, wherein each of the therapy metrics are calculated using a cost function analysis by:
determining a first partial therapy metric associated with the non-linear response structure using a non-linear function;
determining a second partial therapy metric associated with the set of linear response voxels using a linear function; and
summing the first partial therapy metric and the second partial therapy metric to determine the first therapy metric.

7. The system of claim 1, wherein the optimizer is configured to determine, for each of a plurality of total current amplitudes, at least a first therapy metric for the non-linear voxels using a non-linear function by determining a quantity of voxels in the non-linear response structure are activated in a given steering configuration and at a given total current amplitude, and applying a non-linear function to the quantity.

8. The system of claim 7, wherein the non-linear function is a polynomial function.

9. The system of claim 7, wherein the non-linear function is a step function.

10. The system of claim 7, wherein the non-linear function comprises two or more segments, each segment corresponding to a range of the quantity, and each segment applying a different linear or non-linear function.

11. The system of claim 1, wherein the optimizer is configured to estimate voltage fields within the first non-linear structure to determine whether the non-linear structure would be activated at a given steering configuration and total current amplitude.

12. The system of claim 1, wherein the therapy selection module is configured to generate and display an image to the user to aid in selecting among the candidate therapies, the image indicating a volume of activation of the neural tissue for at least one candidate therapy.

13. The system of claim 1, further comprising a communications circuit configured to communicate a selected therapy candidate to be tested to a pulse generator of the neurostimulation system, the pulse generator being connected to the lead to allow delivery electrical outputs defined by the selected therapy candidate to the patient.

14. The system of claim 1, wherein the first non-linear response structure is a nerve fiber or a bundle of nerve fibers.

15. The system of claim 1, wherein the first non-linear response structure is a nerve fiber located in the internal capsule, and the lead is positioned to deliver therapy to a target in the thalamus.

16. A method of configuring a neurostimulations system comprising:
receiving, in a computing system, brain anatomy data for a patient and lead position data for a lead implanted in the brain of the patient;
defining volume portions of the patient's brain as voxels surrounding the lead, including identifying non-linear voxels associated with a first non-linear response structure in the brain, and to treating other voxels outside of the first non-linear response structure as linear voxels;
presenting, via a user interface, a graphical output allowing a user to identify and select structures in the patient's brain as target structures and as avoid structures;
identifying a plurality of candidate therapies by:
a) selecting a steering configuration for simulating issuance of output current in a fractional manner across a plurality of electrodes on the lead, the electrodes each receiving a fraction of a total current;
b) calculating, for the selected steering configuration, and for each of a plurality of total current amplitudes, at least a first therapy metric for the non-linear voxels using a non-linear function, and a second therapy metric for the linear voxels, and summing the first therapy metric with the second therapy metric; and
c) selecting a different steering configuration and repeating a) and b) to generate a plurality of therapy candidates each identifying a therapy metric associated with a particular total current amplitude and steering configuration; and
presenting to a user, via the graphical user interface, the candidate therapies to a user for selection of one or more candidate therapies for testing on a patient.

17. The method of claim 16, further comprising associating each target structure and each avoid structure with a weight to be used in calculating the therapy metrics.

18. The method of claim 17, wherein the first non-linear response structure is a nerve fiber, and a weight associated with the first non-linear response structure is calculated by determining neural structures to which the nerve fiber connects.

19. The method of claim 16, further comprising associating a background weight with any volume in the patient neural tissue that would be activated by a therapy, and using the background weight to determine a background penalty when calculating the therapy metrics.

20. The method of claim 16, further comprising selecting the candidate therapies by identifying a combination of total current amplitude and highest metric for each steering configuration, and selecting the combinations of steering configuration and total current amplitude having highest metrics as the candidate therapies.
